(19) United States
(12) Patent Application Publication
(10) Pub. No.: US 2025/0295902 A1
(43) Pub. Date: Sep. 25, 2025

(54) Microneedle

(71) Applicant: University College Dublin, National University of Ireland, Dublin, Dublin (IE)

(72) Inventors: Jufan Zhang, Dublin 18 (IE); Fengzhou Fang, Dublin 4 (IE); Lorcan O'Toole, Dublin 16 (IE)

(21) Appl. No.: 18/863,387

(22) PCT Filed: May 9, 2023
(86) PCT No.: PCT/EP2023/062212

§ 371 Date: Nov. 6, 2024

(30) Foreign Application Priority Data
May 9, 2022 (GB) 2206750.8

(51) Int. Cl. A61M 37/00

(52) U.S. Cl. CPC A61M 37/0015; A61M 2037/0023; A61M 2037/0038; A61M 2037/0053

(57) ABSTRACT

A microneedle for transdermal drug delivery, the microneedle comprising an input channel extending through the microneedle along a longitudinal axis of the microneedle, the input channel defining a sidewall, a first end, and a second end, the input channel configured to receive fluid input into the microneedle. The microneedle comprises one or more outlet channels extending between an interior surface of the sidewall, and an exterior surface of the sidewall, such that each of the one or more outlet channels define a fluid path between the input channel and the exterior surface of the sidewall. The one or more outlet channels are angled relative to the longitudinal axis of the microneedle at an angle which is greater than 0° and less than 90°.

### FIELD OF INVENTION

The invention relates generally to microneedles. More particularly, but not exclusively, the invention relates to microneedles for transdermal drug delivery.

### BACKGROUND

Conventionally, medicaments are delivered into a human body orally, using hypodermic needles, or through other means such as pulmonary drug delivery devices.

The oral delivery of medicaments into the human body is problematic, mainly due to the degradation of the medicaments in the gastrointestinal tract. Drugs that are administered orally (as opposed to intravenously, intramuscularly, sublingually, or transdermally) must first pass from the intestine to the liver before reaching general circulation. Thus, for many drugs, the dose is reduced by xenobiotic metabolism before reaching the body's tissues. Some drugs are metabolized by gut flora or digestive enzymes.

Further, the delivery of medicaments using hypodermic needles is often painful for patients and it is not appropriate for long-term, continuous deliveries or self-administration. Many patients suffer from a phobia of needles, making it difficult for a medical professional to inject the patient. Injection of hypodermic needles often leads to bruising, and infections may occur at the point of insertion of the needle.

An alternative method for the delivery of medicaments into the human body is through the use of a transdermal patch. However, medicament delivery through transdermal patches is severely limited by the inability of a large majority of medicaments to enter the body through the skin at therapeutic rates. Medicament delivery rates are limited by the skin's outer layer (the stratum corneum), which is approximately 10-30 μm thick and is composed of keratinized dead cells and scales. Skin permeability is increased enormously if the stratum corneum layer is disrupted. To disrupt the layer, a number of different approaches have been studied, ranging from chemical/lipid enhancers, to electric fields employing iontophoresis and electroporation, to pressure waves generated by ultrasound or photoacoustic effects. These enhancement methods have only had a limited impact on medical practices to date. Chemical methods can negatively affect the skin and the medicaments being delivered. Further, the other methods require the use of complex systems.

Microneedles are effective in forming micro perforations in the stratum corneum. Further, they do not chemically react with the skin or the medicament being delivered. Therefore, microneedles are seen as promising, minimally invasive drug delivery devices which act as alternatives to pills/tablets, conventional needles, and transdermal patches. The reduced size of a microneedle (typical at dozens to hundreds of micrometres in width and length) compared with a conventional hypodermic needle reduces, or eliminates, the pain experienced by a patient during treatment, such as when used for intravenous injection and intramuscular injections.

Currently, the biggest issue associated with microneedle drug delivery is that the applicable dose of medicament is too small. Typically, most current microneedles are configured to deliver small dosage, such as between 0.3 ml and 1 ml, of medicament to a patient, and the dosage is usually limited below 2 ml. As such, it is difficult to deliver therapeutic levels of medicament using current microneedle technology. Simply increasing the number of microneedles in use at one time causes a significant rise in the difficulties and costs associated with production of microneedle devices, and cannot solve the problem of fast saturation of skin caused by delivering drug liquid into a small region of the same skin layer simultaneously.

Hollow microneedles allow for continuous delivery of drug liquid by virtue of the existence of the microchannel inside. The complex design of hollow microneedles leads to manufacturing difficulties, and cost-effective manufacturing is still a major challenge for producers of microneedle devices. In view of the high costs and difficulties associated with manufacturing, along with the concerns on potential fast saturation of the injected skin layer, most hollow microneedle arrays comprise a limited number of microneedles, such as 1, 3 or 12 microneedles, as has been adopted by commercial systems.

The present invention has been devised with the foregoing in mind.

### SUMMARY OF INVENTION

According to an aspect of the invention, there is provided a microneedle for transdermal drug delivery. The microneedle comprises an input channel extending through the microneedle along a longitudinal axis of the microneedle. The input channel defines a sidewall, a first end, and a second end. The input channel is configured to receive fluid input into the microneedle. The fluid may be a liquid, such as a liquid medicament. The longitudinal axis extends between the first and second end of the microneedle.

The microneedle comprises one or more outlet channels. Each of the one or more outlet channels may define a fluid path between the input channel and an exterior surface of the microneedle.

The outlet channels may extend between an interior surface of the sidewall and an exterior surface of the sidewall. The exterior surface of the sidewall may be the exterior surface of the microneedle. Each of the one or more outlet channels may define a fluid path between the input channel and the exterior surface of the sidewall.

The one or more outlet channels are angled relative to the longitudinal axis of the microneedle at an angle which is greater than 0° and less than 90°. In this way, the microneedle may be configured such that fluid enters the input channel travelling along an input direction pointing along the longitudinal axis (i.e. at 0°) the outlet channels are angled relative to this input direction at an angle which is greater than 0° and less than 90° to the input direction.

The outlet channels may be formed in the sidewall, rather than at the second end of the microneedle. Providing outlet channels in the sidewall reduces the hydrostatic pressure which fluid being delivered via the microneedle needs to overcome to enter a patient's body. Further it reduces the high resistance applied by the skin at the needle tip which impedes drug delivery when having outlet channels in the second end of the microneedle. As such, having outlet channels formed in the sidewall of the microneedle improves the drug delivery rate of the microneedle. Outlet channels formed in the sidewall may be referred to as lateral outlet channels.

The outlet channels are angled relative to the longitudinal axis at an angle greater than 0° but less than 90°. As fluid passes from the input channel into the one or more outlet channels it changes flow direction. If the outlet channel was angled at 90°, i.e., perpendicular to the longitudinal axis of the microneedle, the fluid would undergo an abrupt change. Abrupt changes in fluid flow direction can cause blockages and reduce fluid flow rate potentially. Using an off-axis angled outlet channel relative to the longitudinal axis reduces the variation in flow direction. As such, this arrangement helps to reduce blockages and maintain a greater fluid flow rate when compared with outlet channels angled at 90° relative to the longitudinal axis.

An outlet channel angled at 0° relative to the longitudinal axis of the microneedle would run parallel with the longitudinal axis and would be formed in an end of the microneedle (such as the second end) rather than the sidewall. As explained above, such a microneedle would require fluids to overcome a greater resistance (for example, due to hydrostatic pressure differences) when entering into a patient when compared with outlet channels which are formed in the sidewall and are angled at greater than 0° with respect to the longitudinal axis of the microneedle.

The angle between the outlet channel and the longitudinal axis may be between 10° and 70°. The angle between the outlet channel and the longitudinal axis may be between 20° and 60°. The angle may be greater than 45°.

The microneedle may comprise one or more outlet channels disposed in the sidewall as described above, as well as a vertical outlet channel disposed at the tip of the needle.

The fluid may be received through the first end of the input channel. The second end of the input channel may be distal to the first end of the microneedle and the first end of the input channel may be proximal to the first end of the microneedle.

The outlet channels may have curved edges. Having a curved edge makes the change in direction of the fluid flow less abrupt, which may help reduce the chance of blockages and increase the fluid flow rate.

The first end of the input channel may be open. The open first end of the input channel may be configured such that fluids may enter the input channel through the first end of the input channel.

The second end of the input channel may be closed. The closed second end may prevent fluids passing therethrough. A needle tip may be disposed on the second, closed end of the inlet channel. The needle tip may be configured to penetrate a patient's skin. In embodiments where a needle tip is disposed on the second end of the input channel, the second end of the input channel is not the end of the microneedle. In other arrangements, the second end of the input channel may be the second end of the microneedle. The first end of the input channel may be the first end of the microneedle.

The microneedle may comprise a plurality of outlet channels. The positions of the outlet channels on a microneedle may be referred to as the pattern (of the outlet).

Having a plurality of outlet channels increases the drug delivery rate as there are more pathways through which liquid drugs can enter the body. Furthermore, the total area for outletting the drug liquid can be controlled using different numbers of outlet channels. Also, if one of the outlet channels is blocked, the microneedle is still operable.

The outlet channels may be off-set relative to each other along the longitudinal axis of the microneedle. The outlet channels may be angularly off-set relative to each other around the longitudinal axis of the microneedle. The outlet channels may be angularly off-set relative to each other evenly around the longitudinal axis, such that the outlet channels are evenly distributed around the longitudinal axis.

Having outlet channels at different positions along the microneedle helps to reduce the effects of saturation. Once a region of the body has taken up/absorbed a certain amount of the liquid drug without sufficient diffusion outwards, it cannot absorb anymore, and any further drugs being delivered to the area will be ineffectual. Staggering the positions of the outlet channels along the longitudinal axis and angularly around the longitudinal axis ensures that each outlet channel delivers fluid to a different part of the skin. For example, the outlet channels may deliver fluids to different layers of skin. Furthermore, having multiple outlets which are angularly offset from each other around the longitudinal axis directs the liquid medicament in different directions. This controls the diffusion of the drugs in the skin in order to avoid over-saturation.

The presence of outlet channels may reduce the structural strength of the microneedle. Evenly distributing the outlet channels angularly around the longitudinal axis improves the structural strength of the microneedle relative to a microneedle where the outlet channels are grouped together. Off-setting the outlet channels along the longitudinal axis of the microneedle prevents the formation of weak areas of the microneedle which are prone to breaking and/or snapping.

A width of the input channel may vary along the longitudinal axis of the microneedle. The width of the input channel may be constant along the longitudinal axis of the microneedle. The input channel may have a tapered profile, a sinusoidal profile, a staggered profile, a stepped profile, or an irregular profile.

Having a 3D internal structure allows a designer to optimise fluid flow based on outlet channel positions. The 3D internal structure refers to the changing/unchanging profile of the input channel and the changing/unchanging profile of the outlet channel. The variation in width of the input channel can be used to regulate the pressure drop and fluid velocity throughout the input channel. Varying the width of the input channel also allows a designer to enlarge or narrow the drug delivery path relative to a microneedle with an input channel with a constant width.

The width of the microneedle may vary along the longitudinal axis. The varying width of the microneedle may define the shape of the microneedle. The microneedle may be a cylinder, a tapered cylinder, a pyramid, a tetrahedron, or a cone. The shape of the microneedle may be configured to increase the strength and durability of the microneedle to reduce the likelihood of the microneedle breaking.

The microneedle may be formed from, or comprise, a polymeric material. The microneedle may be formed from, or comprise, high-strength bio-compatible polymeric material, such as Polyglycolide (PGA), Polylactic acid (PLA), Polymethyl methacrylate (PMMA), Cyclic olefin copolymer (COC), Polycarbonate (PC), or Liquid crystal polymer (LCP).

Alternatively, or in addition, the microneedle may be formed from, or comprises, metal, ceramic, or a semiconductor.

In some arrangements, the one or more outlet channels comprise a first end formed in the interior surface of the sidewall, and a second end formed in the exterior surface of the sidewall. One or more of the one or more outlet channels may be tapered, such that the size of the first end of the one or more outlet channels is not equal to the size of the second end of the outlet channel.

The size of the first end of the one or more outlet channels and the size of the second end of the one or more outlet channels may be the cross-sectional size. The first end of the outlet channel may be larger than the second end of the outlet channel. Alternatively, the second end of the outlet channel may be larger than the first end of the outlet channel. The outlet channel may be smoothly tapered, such that the size of the outlet channel varies gradually between the first and second end. The outlet channel may have a 3D profiled internal structure between the first and second ends, such that the size of the outlet channel varies between the first and second end.

Having tapered outlet channels allows a microneedle to be configured to increase or decrease the cross-sectional area of flow pathway in the outlet channel, for example to influence flow speed. This flexibility enables the manufacture of microneedles customised for specific applications. The variation in width of the outlet channel can be used to regulate the pressure drops and fluid velocity.

The microneedle may comprise a mixture of tapered and non-tapered outlet channels. The microneedle may only comprise tapered outlet channels. The microneedle may not comprise any tapered outlet channels.

According to a further aspect of the invention, there is provided a microneedle array device. The microneedle array device comprises a base. The microneedle array device comprises one or more microneedles. The microneedles may be the microneedles of the above-described aspect of the invention. The one or more microneedles may be disposed on a first side of the base. The microneedle array device comprises a drug inlet. The drug inlet may be disposed on a second side of the base. The microneedle array device may comprise a hollow chamber inside the base. The hollow chamber may form a fluid connection between the one or more microneedles and the drug inlet.

The microneedle array device allows a clinician, other medical professional, or patient to easily administer fluids, such as liquid drugs/medicaments, into the body. Fluids can be conveyed into and through the microneedle array device via the drug inlet using a syringe or other device. The one or more microneedles are sufficiently small to easily penetrate a patient's skin without causing much pain or discomfort.

The first side of the base may be opposite to the second side of the base. Alternatively, the first side of the base may be adjoining with the second side.

The hollow chamber may connect the first side of the microneedle array device to the second side. The hollow chamber may provide a fluid pathway between the drug inlet on the first side and the input channels of the microneedles located at the second side.

The drug inlet may comprise connection means. The connection means may connect the microneedle array device to a syringe or receptacle. The connections means may be, or comprise, a tapered hole. The connection means may be, or comprise, an elastic sealing ring, or other types of sealing components.

The connection means reduces the chance of the syringe or other device from slipping out of position whilst fluids are being conveyed into the microneedle array device. This makes it easier for a user to inject the fluids into the patient. This makes the microneedle array device more flexible when connected with an external device. Further, it enables the microneedle array device to connect with various external devices, such as syringes. The connection means reduces the chance of fluid leakage when injecting drugs into the body.

The one or more of the microneedles may comprise a first microneedle comprising one or more outlet channels and a second microneedle comprising one or more outlet channels.

The one or more outlet channels of the first microneedle may be distributed in a first pattern and the one or more outlet channels of the second microneedle may be distributed in a second pattern.

The first pattern may be different to the second pattern.

As described above, if fluids are delivered to the same region of the body, fluid uptake is reduced by saturation. Using different outlet channel patterns between different microneedles allows fluid delivery to a range of different areas to avoid saturation effects.

One of the one or more outlet channels distributed in the first pattern may have a different position along the longitudinal axis of microneedle to one of the one or more outlet channels distributed in the second pattern.

One or more of the outlet channels in the first pattern may have the same position along the longitudinal axis as one or more of the outlet channels of the second pattern.

One of the one or more outlet channels distributed in the first pattern may have a different position along the longitudinal axis of microneedle to each of the one of the one or more outlet channels distributed in the second pattern.

Each of the one or more outlet channels distributed in the first pattern may have a different position along the longitudinal axis of microneedle to each of the one of the one or more outlet channels distributed in the second pattern.

One of the one or more outlet channels distributed in the first pattern may have a different angular position around the longitudinal axis of microneedle to one of the one or more outlet channels distributed in the second pattern.

One of the one or more outlet channels distributed in the first pattern may have a different angular position around the longitudinal axis of microneedle to each of the one or more outlet channels distributed in the second pattern.

One or more of the outlet channels in the first pattern may have the same angular position around the longitudinal axis as one or more of the outlet channels of the second pattern.

Each of the one or more outlet channels distributed in the first pattern may have a different angular position around the longitudinal axis of microneedle to each of the one or more outlet channels distributed in the second pattern.

One of the one or more microneedles may comprise one or more outlet channels distributed in the same pattern as the outlet channels of one or more of the other microneedles. Alternatively, each microneedle of the one or microneedles may comprise a unique outlet channel pattern.

A first microneedle of the plurality of microneedles may have a different length to at least one other microneedle in the plurality of microneedles. Each microneedle of the plurality of microneedles may have a different length to each other. In some embodiments, every microneedle in the microneedle array device may have the same length.

Having microneedles of various lengths allows a microneedle array device to form an array of microneedles with a profiled surface pattern. The surface pattern can be designed to match the shape/surface of a patient's skin.

In some arrangements, microneedles positioned in a central region of the microneedle array device may be longer than microneedles positioned in an outer region, such that the surface pattern defines a protruding region. In other arrangements, microneedles positioned in an outer region of the microneedle array device may be longer than microneedles positioned in a central region, such that the surface pattern defines an indented region.

Further advantageously, having different length microneedles enables the microneedles to penetrate to different depths in the patient to ensure fluids are delivered to desired regions and to reduce the chance of saturation occurring at a single depth. In addition, different length microneedles may match with a human body's profile more perfectly and prevent leaking from the patient due to over saturation of the patient tissue or insufficient penetration of microneedles in some regions.

According to a further aspect of the invention, there is provided a method of manufacturing a microneedle. The method may be used to manufacture the microneedle of the above aspect of the invention, or the microneedle array device of the above aspect of the invention.

The method may include providing a mould. The method may include providing a polymeric material. The method may include injection moulding the polymeric material using the mould to form the microneedle.

Injection moulding enables a manufacturer to mass produce polymer microneedles, in a cost-effective and efficient way.

Advantageously, the injection moulding process to produce a microneedle array device enables a manufacturer to produce a microneedle array device with integral microneedles.

The method may comprise forming one or more outlet channels during the step of injection moulding.

Forming outlet channels during the step of injection moulding reduces the complexity associated with manufacture compared with some other traditional manufacturing processes. The microneedles can be produced without requiring further steps.

The method may further comprise the step of removing one or more sections of the sidewall so as to form the one or more outlet channels of the microneedle. This step may be carried out after the step of injection moulding. Removing one or more sections of the sidewall may comprise laser cutting. Removing one or more sections of the sidewall may comprise direct polymer cutting. Removing one or more sections may comprise any other suitable method.

Forming microneedles without outlet channels during the step of injection moulding reduces complexity of the injection moulding process. This allows manufacturers to use simple moulds. Further, it enables manufacturers to mass produce microneedles using the same mould, as the outlet channels can be added afterwards.

Due to the low cost of injection moulding, any device comprising microneedles manufactured using the method of this aspect can be disposable (for one-time use) when necessary. Thorough cleaning of microneedle devices is difficult due to their small size and complex structure, so cleaning and sterilization of microneedle devices may not be practical for the self-administration devices used by some patients, and so there is a demand for disposable, one-time use microneedle devices.

According to a further aspect of the invention, there is provided a microneedle for transdermal drug delivery. The microneedle comprises an input channel extending through the microneedle along a longitudinal axis of the microneedle. The input channel defines a sidewall, a first end, and a second end. The input channel is configured to receive fluid input into the microneedle. The fluid may be a liquid, such as a liquid medicament. The longitudinal axis extends between the first and second end of the microneedle. The microneedle comprises one or more outlet channels. The outlet channels may extend between an interior surface of the sidewall and an exterior surface of the sidewall. The one or more outlet channels may comprise a first end formed in the interior surface of the sidewall, and a second end formed in the exterior surface of the sidewall. One or more of the one or more outlet channels may be tapered, such that the size of the first end of the one or more outlet channels is not equal to the size of the second end of the outlet channel.

The size of the first end of the one or more outlet channels and the size of the second end of the one or more outlet channels may be the cross-sectional size. The first end of the outlet channel may be larger than the second end of the outlet channel. Alternatively, the second end of the outlet channel may be larger than the first end of the outlet channel. The outlet channel may be smoothly tapered, such that the size of the outlet channel varies gradually between the first and second end. The outlet channel may have a 3D profiled internal structure between the first and second ends, such that the size of the outlet channel varies between the first and second end.

Having tapered outlet channels allows a microneedle to be configured to increase or decrease the cross-sectional area of a flow pathway in the outlet channel, for example to influence flow speed. This flexibility enables the manufacture of microneedles customised for specific applications. The variation in width of the outlet channel can be used to regulate the pressure drops and fluid velocity

The microneedle may comprise a mixture of tapered and non-tapered outlet channels. The microneedle may only comprise tapered outlet channels. The microneedle may not comprise any tapered outlet channels.

Optional features of any of the above aspects may be combined with the features of any other aspect, in any combination. For example, features described in connection with the microneedle of the above aspect may have corresponding features definable with respect to the method of the above aspect, and vice versa, and these embodiments are specifically envisaged. The microneedle array device of one aspect of the invention may comprise one or more microneedles according to any other aspect of the invention. Features which are described in the context or separate aspects and embodiments of the invention may be used together and/or be interchangeable wherever possible. Similarly, where features are, for brevity, described in the context of a single embodiment, those features may also be provided separately or in any suitable sub-combination.

### BRIEF DESCRIPTION OF THE DRAWINGS

The invention will now be described, by way of example only, with reference to the accompanying drawings, in which:
FIG. 1 shows a perspective side view of a microneedle, showing internal structure of the microneedle, according to the present invention;

FIG. 2 shows a perspective side view of another microneedle, showing internal structure of the microneedle, according to the present invention;

FIGS. 3a and b respectively show perspective side views of another microneedle according to the present invention, with FIG. 3b showing internal structure of the microneedle;

FIGS. 4a and b show side views of a microneedle, FIG. 4c shows a side view of the microneedle according to the present invention with the positions of the outlet channels clearly indicated;

FIGS. 5a and 5b show side views of another microneedle according to the present invention, with FIG. 5a showing internal structure of the microneedle;

FIG. 6a shows a perspective side view of a microneedle according to the present invention showing the internal structure of the microneedle, FIG. 6b shows a plan view of the microneedle of FIG. 6a;

FIG. 6c shows a perspective side view of a microneedle, showing the internal structure of the microneedle, according to the present invention;

FIGS. 7a and b respectively show views of first and second sides of a microneedle array device according to the present invention;

FIG. 8 shows a cross-sectional illustrative view of a microneedle array device according to the present invention;

FIG. 9 shows a cross-sectional illustrative view of a microneedle array device comprising attachment means according to the present invention;

FIG. 10 shows a cross-sectional illustrative view of a microneedle array device according to the present invention comprising microneedles with outlet channels arranged according to different patterns;

FIG. 11 shows a cross-sectional illustrative view of a microneedle array device according to the present invention comprising microneedles with different lengths;

FIG. 12 shows a microneedle array device in accordance with the present invention comprising two types of microneedles;

FIGS. 13a and b show perspective and side views, respectively, of the types of microneedles of the microneedle array device of FIG. 12 in greater detail;

FIG. 14 shows a plan view of the microneedle array device of FIG. 12 indicating the outlet facing directions;

FIG. 15 shows an overview flow diagram of a method for manufacturing microneedles and microneedle array devices;

FIG. 16 shows a perspective view of a microneedle array device according to the present invention;

FIGS. 17a and b show schematic side views of microneedle array devices according to the present invention; and

FIGS. 18a and b show schematic side views of microneedle array devices according to the present invention.

### DETAILED DESCRIPTION

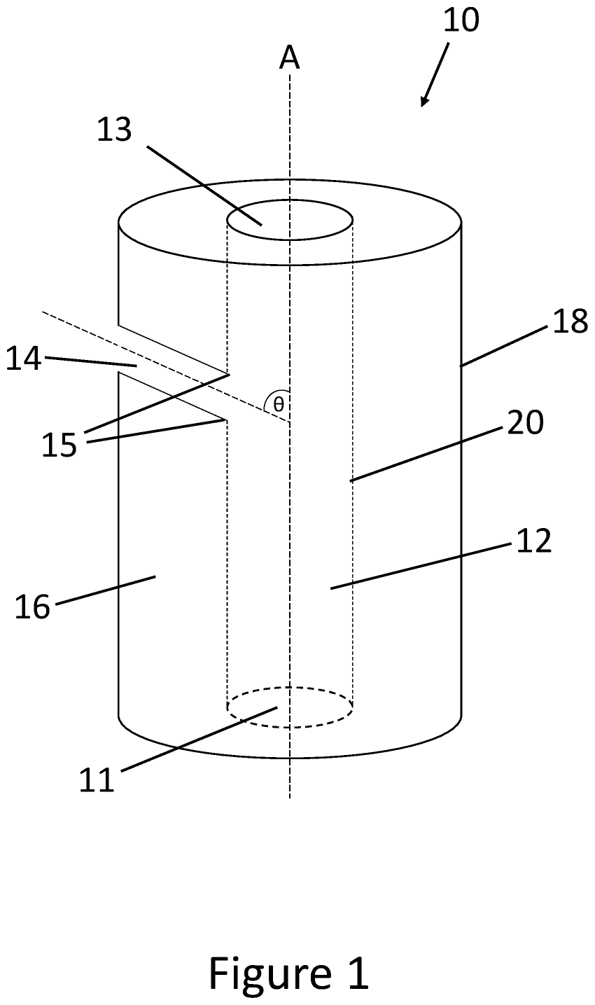

FIG. 1 shows a perspective view of a microneedle 10. The microneedle 10 comprises an input channel 12 extending through the microneedle 10 along a longitudinal axis (A) of the microneedle 10. The input channel 12 defines a sidewall 16, a first end 11, and a second end 13. The longitudinal axis (A) of the microneedle 10 is the longitudinal axis of the input channel 12.

The sidewall 16 comprises an interior surface 20 and an exterior surface 18. The exterior surface 18 of the sidewall 16 is the exterior surface of the microneedle 10. The interior surface 20 of the sidewall 16 defines the width of the input channel 12. In the embodiment of FIG. 1, the exterior surface 18 is concentric with the interior surface 20. In other embodiments, the exterior surface 18 may not be concentric with the interior surface 20. In the embodiment of FIG. 1, the width of the sidewall 16 is uniform across the entire microneedle 10. In other embodiments, the width of the sidewall 16 varies in different parts of the microneedle 10. For example, in some embodiments, the width of the sidewall 16 varies along the longitudinal axis (A) of the microneedle 10.

In FIG. 1, the input channel 12 is a hollow input channel. In other embodiments, the input channel 12 may not be totally hollow. For example, the input channel 12 may comprise one or more support structures. The support structure may be a frame configured to strengthen internally the microneedle.

The input channel 12 is concentric with the exterior surface 18 of the microneedle 10 and runs through the centre of the microneedle 10. In other embodiments, the input channel 12 may be off-centre from the longitudinal axis (A) of the microneedle 10. In the embodiment of FIG. 1, the input channel 12 runs through the entire microneedle 10 such that the length of the input channel 12 is equal to the length of the microneedle 10. In other embodiments, the input channel 12 may only run though a section of the microneedle 10.

The microneedle 10 comprises an outlet channel 14. The outlet channel 14 is a hollow passageway which extends between an interior surface 20 of the sidewall 16 and an exterior surface 18 of the sidewall 16. The outlet channel 14 provides a fluid pathway between the input channel 12 and the exterior surface 18 of the sidewall 16. The outlet channel 14 provides a fluid pathway for conveying fluid from the input channel 12 to outside of the microneedle 10. In use, the microneedle is inserted through a patient's skin and fluids, such as liquid medicament, are conveyed from the input channel 12 through the outlet channel 14 into the patient.

The outlet channel 14 is angled relative to the longitudinal axis (A) of the microneedle 10. More particularly, the longitudinal axis of the outlet channel is angled relative to the longitudinal axis (A) of the microneedle 10. The outlet channel 14 is angled relative to the longitudinal axis (A) at an angle (θ). The angle (θ) is the angle between the longitudinal axis of the outlet channel and the longitudinal axis of the input channel 12. The angle (θ) is greater than 0° and less than 90°. An angle (θ) of 90° would correspond to an outlet channel 14 running parallel with a transverse axis of the microneedle 10, i.e. perpendicular to the longitudinal axis (A) of the microneedle. In some embodiments, the angle (θ) is greater than 45° and less than 90°. In the embodiment of FIG. 1, the angle (θ) is 65°.

Clearly, an outlet channel 14 having an angle of 0° relative to the longitudinal axis (A) of the microneedle 10, would not be in the sidewall 16 and would instead be in the second end 13 of the microneedle 10, otherwise the outlet channel 14 would not create a fluid pathway between the interior 20 and exterior 18 surfaces of the microneedle 10. As explained earlier in the specification, an outlet channel 14 in the second end 13 of the microneedle 10 would require fluids to overcome a greater resistance (such as hydrostatic pressure) when entering into a patient when compared with outlet channels 14 which are formed in the sidewall 16 and are angled at greater than 0° with respect to the longitudinal axis (A) of the microneedle 10.

In the embodiment of FIG. 1, the microneedle 10 comprises a single outlet channel 14. In other embodiments, the microneedle 10 may comprise a plurality of outlet channels 14.

The outlet channel 14 has internal edges 15. The internal edges 15 define the border between the outlet channel 14 and the interior surface 20 of the sidewall 16. In FIG. 1, the edges 15 are sharp, meaning there is an instant change in angular direction between the interior surface 20 of the sidewall 16 and the outlet channel 14. In other embodiments, the edges 15 may be smooth (such as round), meaning there is a gradual change between the outlet channel 14 and the interior surface 20 of the sidewall 16. The smooth edges 15 may be defined as curved edges 15.

The first end 11 of the input channel 12 is open. The open first end 11 is configured to enable fluids to enter the input channel 12. The second end 13 of the input channel 12 is closed. The closed second end 13 is configured to prevent fluids from passing therethrough.

The microneedle 10 is formed from, or comprises, a polymeric material. The microneedle 10 may be formed from, or comprises, a high-strength bio-compatible polymeric material. The microneedle 10 may be formed from, or comprise, one or more of Polyglycolide (PGA), Polylactic acid (PLA), Polymethyl methacrylate (PMMA), Cyclic olefin copolymer (COC), Polycarbonate (PC), and Liquid crystal polymer (LCP). In other embodiments, the microneedle 10 may be formed from, or comprise, a metal, a ceramic, or a semiconductor.

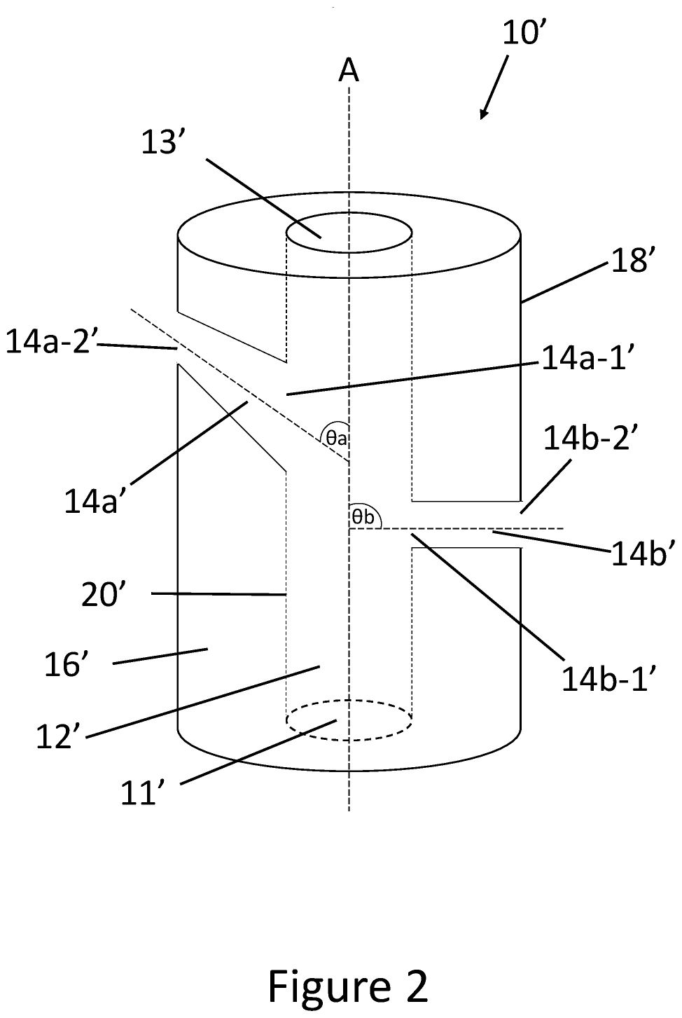

FIG. 2 shows a perspective view of a further microneedle 10′. The microneedle 10′ is substantially identical with the microneedle 10 of FIG. 1, but it includes two outlet channels 14a′, 14b′.

The first outlet channel 14a′ is angled relative to the longitudinal axis (A) of the microneedle 10′ at an angle θa. In FIG. 2, θa is 55°. The first outlet channel 14a′ is tapered, such that a first end 14a-l′ of the outlet channel 14a′ is larger than a second end 14a-2′ of the outlet channel 14a′. The first end 14a-l′ of the outlet channel 14a′ is formed in the interior surface 20′ of the sidewall 16′. The second end 14a-2′ of the outlet channel 14a′ is formed in the exterior surface 18′ of the sidewall 16′. The cross-sectional area of the first end 14a-l′ is greater than the cross-sectional area of the second end 14a-2′. This is in contrast with the outlet channel 14 of microneedle 10 of FIG. 1 for which the outlet channel 14 is not tapered.

When referring to a tapered outlet channel, the angle of that outlet channel relative to the longitudinal axis of the microneedle refers to the angle of a longitudinal axis of the outlet channel extending through the outlet channel relative to the longitudinal axis of the microneedle.

Having an angle θa between 0° and 90° means the first outlet channel 14a′ is angled away from the first end 11′ of the microneedle 10′ and towards the second end 13′ of the microneedle 10′, such that the second end 14a-2′ of the outlet channel 14a′ is further from the first end 11′ of the microneedle 10′ than the first end 14a-l′ of the outlet channel 14a′. An angle θa between 90° and 180° would correspond to an outlet channel 14a′ angled toward the first end 11′ of the microneedle 10′, such that the second end 14a-2′ of the outlet channel 14a′ is closer the first end 11′ of the microneedle 10′ than the first end 14a-l′ of the outlet channel 14a′.

The second outlet channel 14b′ is angled relative to the longitudinal axis (A) of the microneedle 10′ at an angle θb. In FIG. 2, Ob is 90°. The second outlet channel 14b′ is not tapered, such that a first end 14b-1′ of the outlet channel 14b′ is the same size as a second end 14b-2′ of the outlet channel 14b′. The first end 14b-1′ of the outlet channel 14b′ is formed in the interior surface 20′ of the sidewall 16′. The second end 14b-2′ of the outlet channel 14b′ is formed in the exterior surface 18′ of the sidewall 16′. The area of the first end 14b-1′ is equal to the area of the second end 14b-2′.

The microneedle 10′ of FIG. 2 comprises a combination of tapered and non-tapered outlet channels 14a′, 14b′. In other embodiments, all the outlet channels 14a′, 14b′ are tapered, or none of the outlet channels 14a′, 14b′ are tapered.

In FIGS. 1 and 2, the microneedles 10, 10′ are shown without a needle tip for purposes of clarity but it would be understood that such a device would usually include a needle tip.

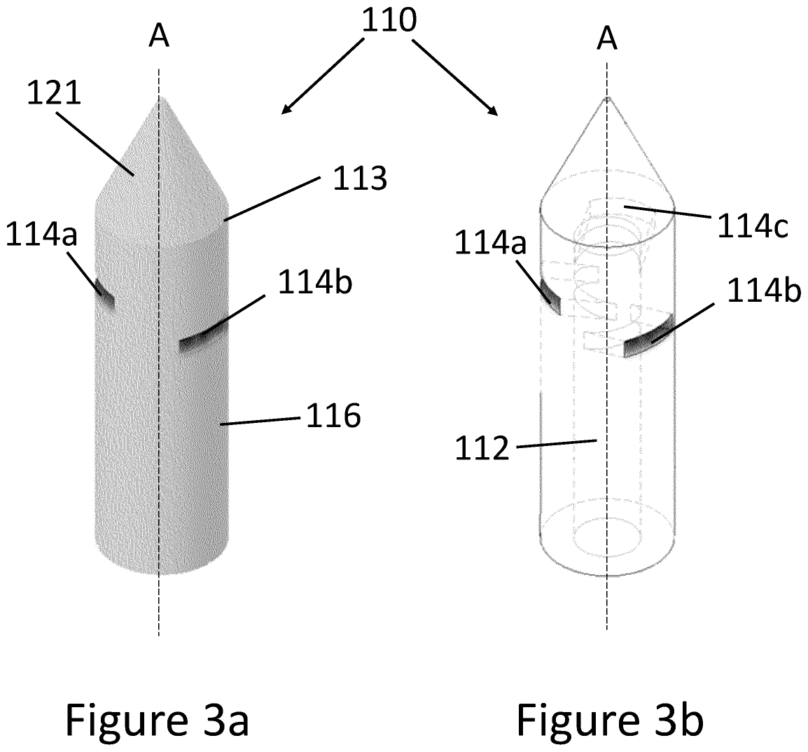

FIGS. 3a and b show perspective views of a microneedle 110. FIG. 3b provides a partially transparent view of the FIG. 3a showing the internal structure. The microneedle 110 corresponds substantially with the microneedle 10 of FIG. 1 except for the number of outlet channels 114a-c.

Microneedle 110 comprises three outlet channels 114a-c. In other embodiments, the microneedle 110 may contain any other number of outlet channels.

The outlet channels 114a-c are off-set relative to each other along the longitudinal axis (A) of the microneedle 110. In the embodiment of FIGS. 3a and b, the outlet channels 114a-c are evenly spaced, such that the distance between consecutive outlet channels 114a-c is consistent. In other embodiments, the spacing between the outlet channels 114a-c may be irregular, such that the distance between consecutive outlet channels 114a-c varies.

In the embodiment of FIGS. 3a and b, the outlet channels 114a-c are also angularly off-set relative to each other around the longitudinal axis (A) of the microneedle 110. The outlet channels 114a-c are angularly off-set evenly around the longitudinal axis (A). There are three outlet channels 114a-c, and the angle between consecutive outlet channels 114a-c is 120° in the arrangement shown in FIG. 3.

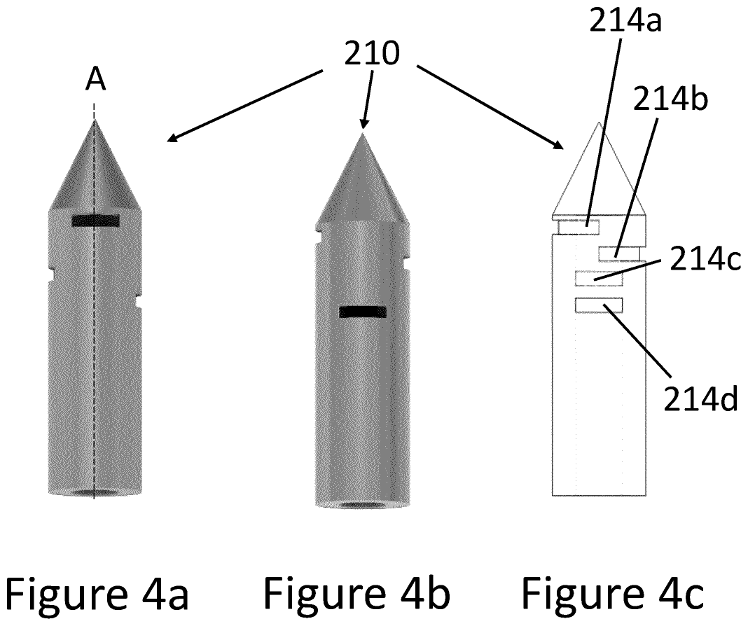

In other embodiments, for example in FIGS. 4a-c where there are four outlet channels 214a-d, the angle around the longitudinal axis between consecutive channels 214a-d is be 90°.

In FIG. 4c, the four outlet channels 214a-d are evenly spaced along the longitudinal axis. The distance between consecutive outlet channels (e.g., distance between 214a and 214b, the distance between 214b and 214c, etc.) is constant.

In other embodiments, the outlet channels 114a-c are not evenly angularly distributed around the longitudinal axis (A) of the microneedle 110. For example, for an embodiment comprising three outlet channels, the first and second outlet channels 114a, b may be angularly off-set from each other by a first angle θ₁, the second and third outlet channels 114b, c may be angularly off-set from each other by a second angle θ₂, and the first and third outlet channels 114a, c may be angularly off-set from each other by a third angle θ₃, wherein the values of θ₁, θ₂ and θ₃ are different from each other.

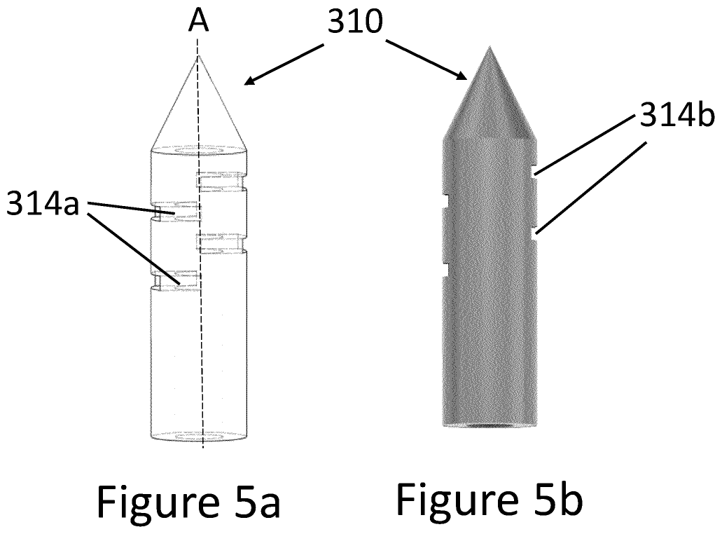

Two or more of the outlet channels 114a-c may be angularly aligned. For example, an outlet channel 114a-c may be aligned with one or more other outlet channels 114a-c and angularly off-set from one or more of the other outlet channels 114a-c. An example of such an embodiment is shown in FIGS. 5a and b. The microneedle 310 comprises four outlet channels 314. A first pair of outlet channels 314a are angularly aligned on a first side of the microneedle 310, and a second pair of outlet channels 314b are angularly aligned on a second side of the microneedle 310. The term “angularly aligned” in this context means that the pair of outlet channels 314 are not displaced from each other angularly around the longitudinal axis (A). The first pair of outlet channels 314a and the second pair of outlet channels 314b are angularly offset relative to each other at 180° around the longitudinal axis (A).

In FIGS. 3a and 3b, each of the outlet channels 114a-c form the same angle (θ) with respect to the longitudinal axis (A). In other embodiments, one or more of the outlet channels 114a-c may form a first angle (θ) with the longitudinal axis (A) of the microneedle 110 and one or more of the other outlet channels 114a-c may form a second, different angle (θ) with the longitudinal axis (A) of the microneedle 110. For example, a first outlet channel may be angled at 50° relative to the longitudinal axis (A) and a second outlet channel may be angled at 45° relative to the longitudinal axis (A). In other embodiments, each outlet channel 114a-c may form a different angle (θ) relative to the longitudinal axis (A).

The microneedle 110 of FIGS. 3a and 3b also comprises a needle tip 121. The needle tip 121 is disposed on the second end 113 of the microneedle 110. The needle tip 121 seals the second end 113 of the input channel 112. In the embodiment of FIGS. 3a and 3b, the needle tip 121 is integrally formed with the sidewall 116 of the microneedle 110. In other embodiments, the needle tip 121 may be a separate component which is connected to sidewall 116. The needle tip 121 forms a sharp point which is sufficiently sharp so as to penetrate a patient's skin. In other embodiments, the microneedle 110 may not comprise a needle tip 121.

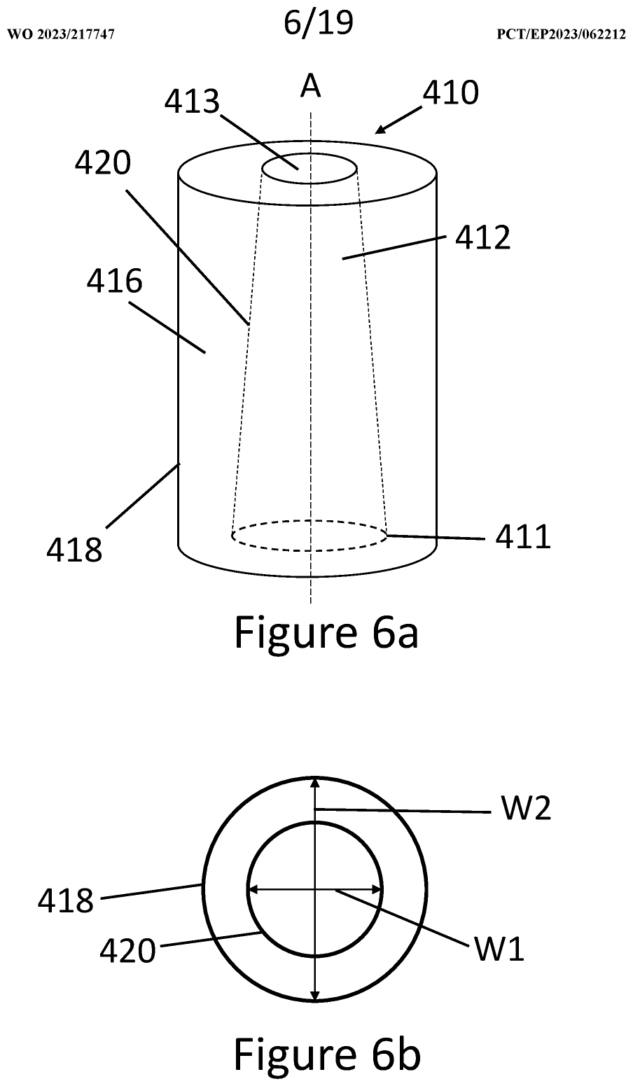

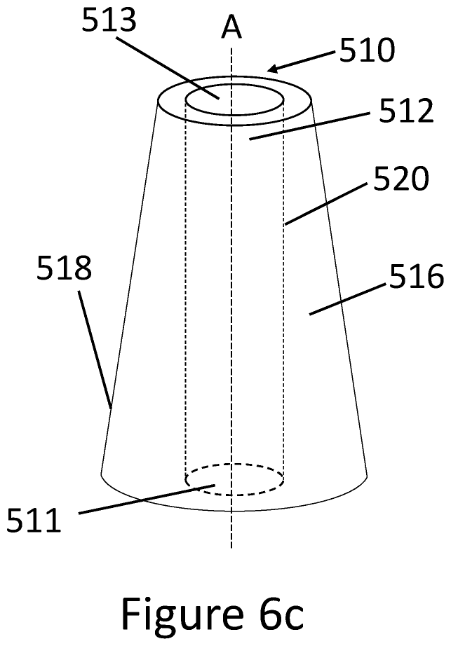

FIGS. 6a and 6c show perspective views of microneedles 410 and 510 according to a further embodiment. FIG. 6b shows a plan view of a cross-section of the microneedle 410 of FIG. 6a. For clarity, the outlet channels and the needle tips are not shown in these figures.

Microneedle 410 comprises an input channel 412 whose width varies along the longitudinal axis (A) of the microneedle 410. The width of the input channel 412 refers to the distance between opposing sides of the interior surface 420 of the sidewall 416 along a transverse plane which is perpendicular to the longitudinal axis (A) of the microneedle 410. The width of the input channel 412 is labelled “W1” in FIG. 6b.

In the example of FIG. 6a, the input channel 412 is tapered towards the second end 413 of the microneedle 410. In other embodiments, the input channel 412 may be tapered towards the first end 411 of the microneedle 410. In other embodiments, the input channel 412 may have a sinusoidal profile, a staggered profile, a stepped profile, or an irregular profile.

The width of the microneedle 410 refers to the distance between opposing sides of the exterior surface 418 of the sidewall 416 along a transverse plane which is perpendicular to the longitudinal axis (A) of the microneedle 410. The width of the microneedle 410 is labelled “W2” in FIG. 6b.

In FIG. 6a, the width of the microneedle 410 is constant. The microneedle 410 is cylindrical because of its constant width.

Microneedle 510 comprises an input channel 512 whose width is constant along the longitudinal axis (A) of the microneedle 510. The width of the microneedle 510 varies along the longitudinal axis (A).

In the example of FIG. 6c, the microneedle 510 is tapered towards the second end 513 of the microneedle 510. In other embodiments, the microneedle 510 may be tapered towards the first end 511 of the microneedle 510. In other embodiments, the microneedle 510 may have a sinusoidal profile, a staggered profile, a stepped profile, or an irregular profile.

The shape of the microneedle 510 is defined by the exterior surface 518 of the sidewall 516. In some embodiments, the shape of the microneedle 510 may be a cylinder (as seen in FIG. 6a), a tapered cylinder (as seen in FIG. 6c), a pyramid, a tetrahedron, a cone or any other shape.

In some embodiments, the width of the microneedle and the width of the input channel both vary with respect to the longitudinal axis of the microneedle.

Experiments were conducted to test the mechanical strength of microneedles formed in accordance with the present invention. A microneedle array device comprising 100 microneedles was used for the experiment. A total force of 22N was applied over the 100 microneedles, resulting in a force of 0.22N being applied to each microneedle. None of the microneedles broke when subject to this force. According to “Park, J.-H., Allen, M. G. and Prausnitz, M. R. Biodegradable polymer microneedles: fabrication, mechanics and transdermal drug delivery. J. Control Release. 104, 51-66 (2005)”, the minimum microneedle strength required for skin penetration is 0.058N.

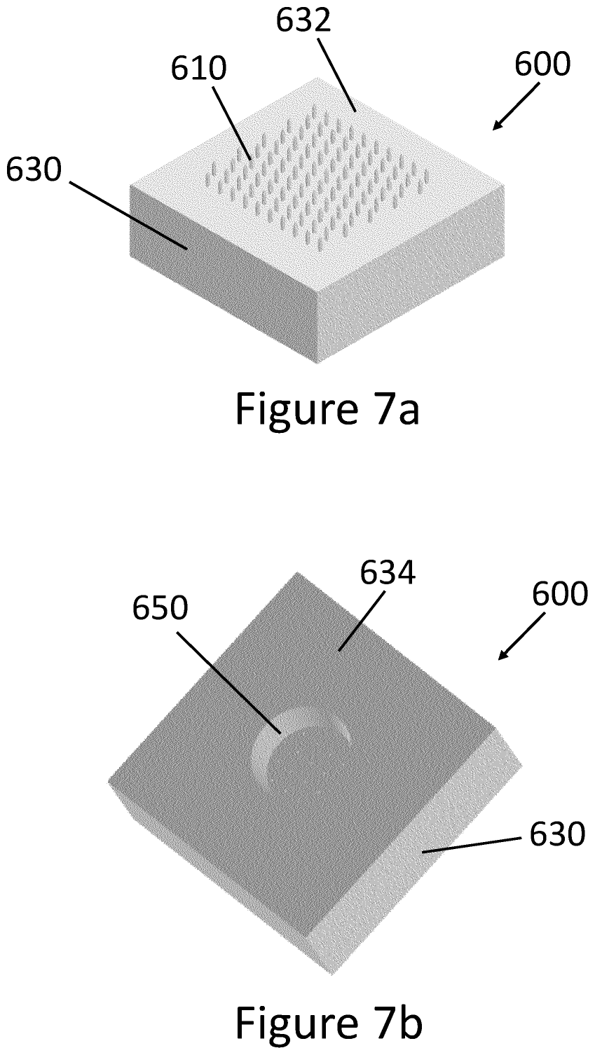

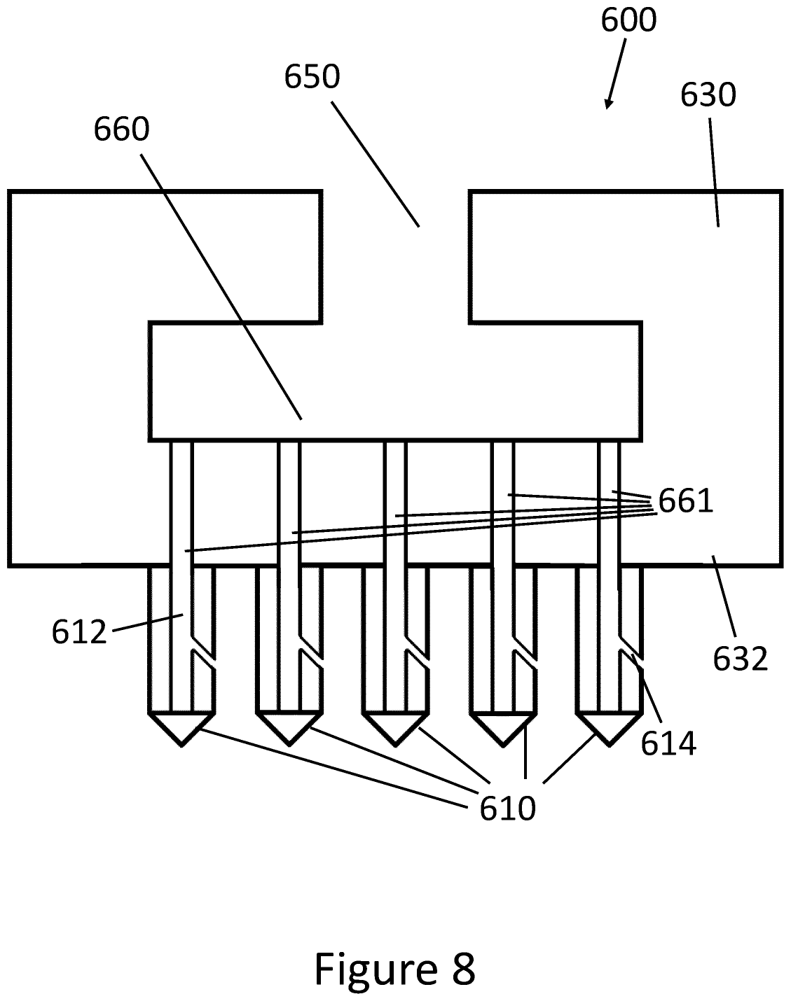

FIGS. 7a and b show first and second sides of a microneedle array device 600. FIG. 8 shows an illustrative cross-sectional side view of the microneedle array device 600. The microneedle array device 600 comprises a base 630, a plurality of microneedles 610, a drug inlet 650, and a hollow chamber 660.

The microneedles 610 are disposed on a first side 632 of the base 630. The number of microneedles can vary in different embodiments. In some embodiments, the microneedle array device 600 may comprise one microneedle 610. In some typical embodiments, the array device may comprise 10-30 microneedles, or in excess of 100 microneedles. The microneedles 610 can be any of the microneedles shown in FIGS. 1-6 and described herein. As shown in FIG. 8, the microneedles 610 are integrally formed with the array device 600. In other embodiments, the microneedles may be connected to the array device 600 without being formed integrally with the array device 600.

The microneedles 610 form a square array in FIGS. 7a and b. In other embodiments, the microneedles may form a circular, rectangular, or triangular array. Further, the microneedle 610 may form an array without a defined shape. The distance between microneedles (centre-to-centre distance between the microneedles) may be uniform or non-uniform.

The drug inlet 650 is disposed on a second side 634 of the base. The second side 634 of the base is opposite the first side 632. In other embodiments, the drug inlet 650 may be disposed on any side of the base.

The hollow chamber 660 is formed inside the base 630. The hollow chamber 660 forms a fluid connection between the one or more microneedles 610 and the drug inlet 650. The hollow chamber 660 forms a continuous fluid pathway between the drug inlet 650 and the microneedles 610. In use, a user inserts one or more fluids through the drug inlet 650 (for example, using a syringe). The hollow chamber 660 provides a fluid pathway which conveys liquids entering through the drug inlet 650 into the input channel(s) 612 of the microneedle(s) 610. The input channels 612 of the microneedles 610 are in fluid connection with fluid channels 661 which extend through the first side 632 of the microneedle array device 600 into the hollow chamber 660. Fluid travels from the hollow chamber 660, through the fluid channels 661 and into the input channels 612 of the microneedles 610. The fluids then exits the microneedles 610 via the outlet channels 614.

In FIG. 8, each microneedle 610 has the same width. In other embodiments, the width of each microneedle 610 may be different from one or more of the other microneedles 610. Each microneedle 610 may have a unique width. In FIG. 8, each microneedle 610 has the same input channel width. In other embodiments, the input channel width of each microneedle 610 may be different from one or more of the other microneedles 610. Each microneedle 610 may have a unique input channel width. In FIG. 8, each microneedle 610 has the same output channel width. In other embodiments, the output channel width of each microneedle 610 may be different from one or more of the other microneedles 610. Each microneedle 610 may have a unique output channel width.

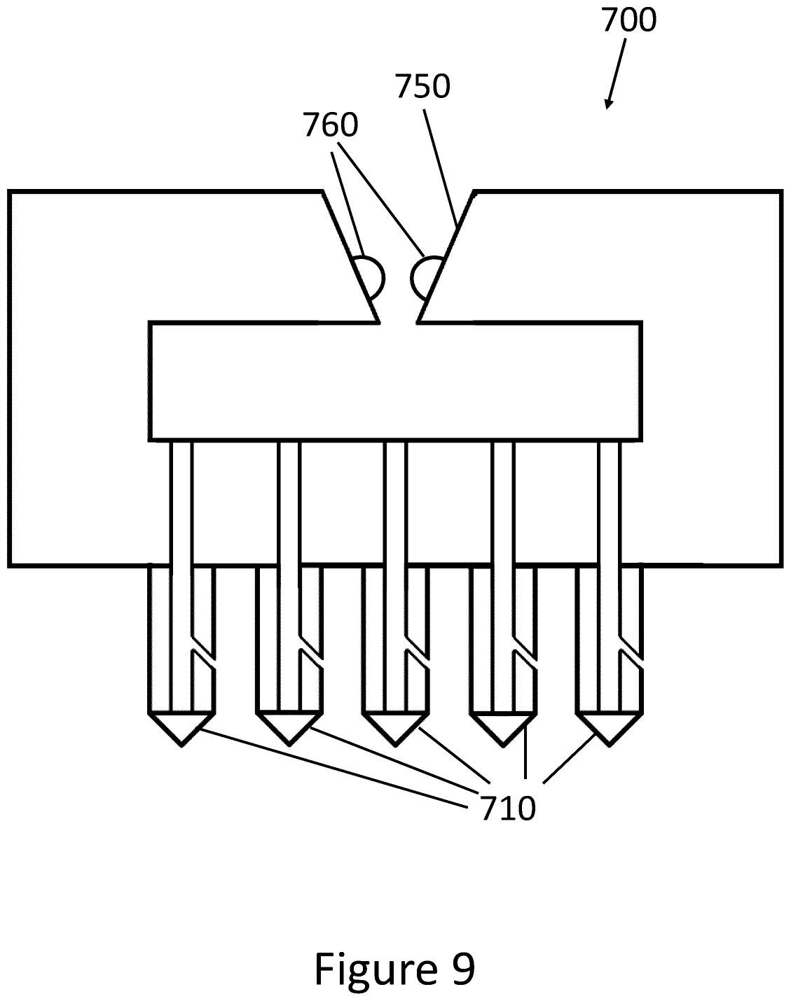

FIG. 9 shows a microneedle array device 700 wherein the drug inlet 750 comprises connection means. The connection means is formed of two components. The first component of the connection means is that the drug inlet 750 has a tapered entrance. In use, as a syringe (or other fluid delivery device) is disposed in the drug inlet 750, the tapered surface abuts the syringe to fix it in place. The drug inlet 750 comprises a second component of the connection means which is an elastic sealing ring 760. The elastic sealing ring 760 is elastically deformable to receive and retain a syringe (or other fluid delivery device), and to prevent leakage of liquid medicament from the microneedle array device 700. Although the two components of the connection means are shown together in FIG. 9, they may be used separately in other embodiments. Further, some embodiments may use one of more of the components of the connection means in combination with further connection means.

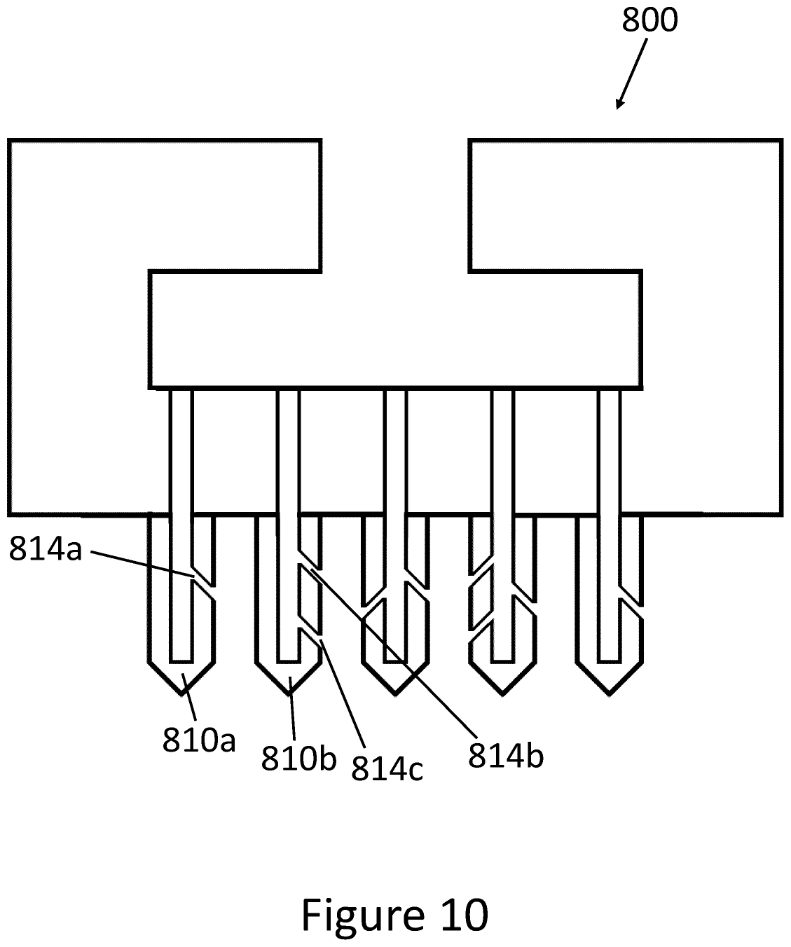

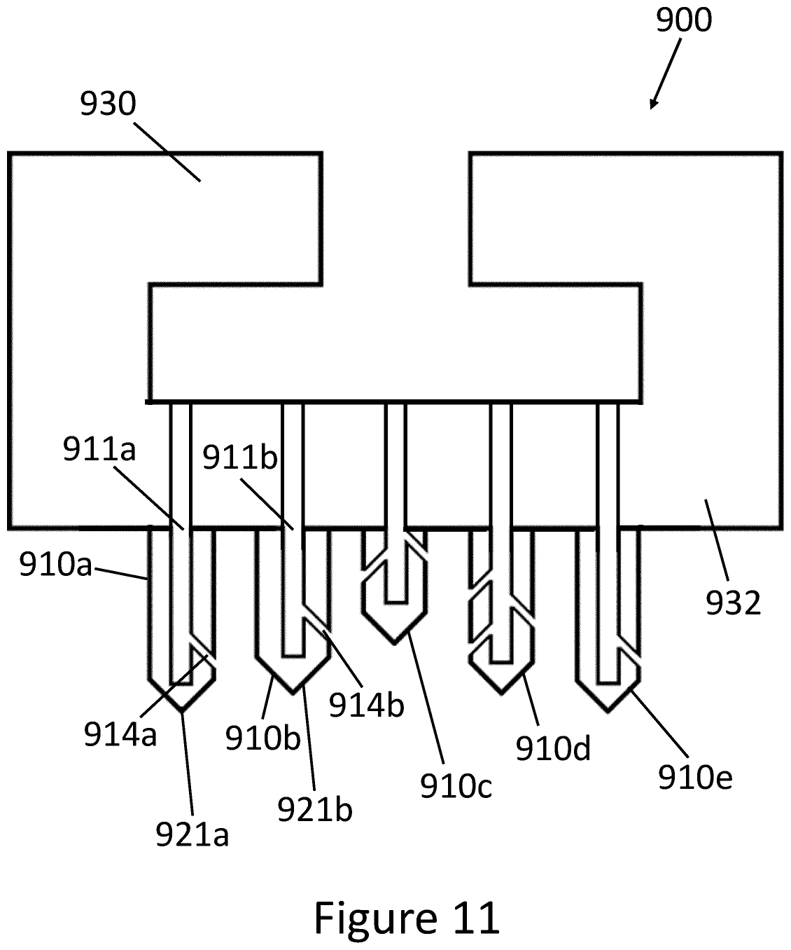

In the microneedle array devices 600, 700 of FIGS. 8 and 9, each microneedle 610, 710 is identical to every other microneedle 610, 710. By contrast, in the microneedle array devices 800, 900 of FIGS. 10 and 11 each microneedle 810, 910 is different to at least one of the other microneedles 810, 910.

In FIG. 10, the number of outlet channels 814a-c varies between microneedles 810. A microneedle 810 within the array device 800 may have a different number of outlet channels 814a-c to one or more of the other microneedles 810 within the array device. In FIG. 10, microneedle 810a comprises one outlet channel 814a, whilst microneedle 810b comprises two outlet channels 814b and c.

In FIG. 10, the position(s) of the outlet channel(s) 814a-c on each microneedle 810 are offset relative to the position(s) of the outlet channel(s) 814a-c of at least one other microneedle 810. The position(s) of the outlet channel(s) 814a-c is unique for each microneedle in the array device 800. The position(s) of the outlet channel(s) 814a-c of the microneedles 810 are be offset along the longitudinal axis of the microneedles 810. The position(s) of the outlet channel(s) 814a-c of the microneedles 810 are angularly offset around the longitudinal axis of the microneedles 810.

In other embodiments, the position(s) of the outlet channel(s) is not unique for each microneedle in the array device 800. For example, for each microneedle, the position(s) of the outlet channel(s) may be identical with one or more other microneedles in the microneedle array device.

In some embodiments, the position(s) of the outlet channel(s) only differs between neighbouring microneedles 810. For example, the position(s) of the outlet channel(s) of a microneedle 810 may differ from the position(s) of the outlet channel(s) of the other microneedles 810 surrounding it. In some embodiments, the position(s) of the outlet channel(s) of the microneedles 810 may form a repeating pattern.

In some embodiments, the outlet channel(s) of each microneedle 810 are positioned such that they do not face an outlet channel of another microneedle 810. The outlet channel(s) of a microneedle may be offset along the longitudinal axis with respect to the outlet channel(s) of neighbouring microneedles 810. The outlet channel(s) of a microneedle may face in a different angular direction about the longitudinal axis to the outlet channel(s) of neighbouring microneedles 810.

FIG. 11 shows a microneedle array device 900 in which the lengths of the microneedles 910a-e are not identical. The length of the microneedle is defined as the distance between the position where the microneedle 911a, b connects to the first side 932 of the base 930 and the needle tip 921a, b. The length of each microneedle 910a-e differs from the length of at least one other microneedle 910a-e. For example, the distance between 911b and 921b is less than the distance between 911a and 921a. In some embodiments, the length of each microneedle 910 differs from the length of every other microneedle 910.

In FIG. 11, the central microneedles 910b-d are shorter than the edge microneedles 910a and e. In other embodiments, the central microneedles 910b-d are longer than the edge microneedles 910a and e. The distribution of lengths of the microneedles 910 across the microneedle array device may form a surface pattern. In some embodiments, the surface pattern may be an angled, pyramidal, or sinusoidal surface pattern. The surface pattern may comprise a protruding portion and/or an indented portion.

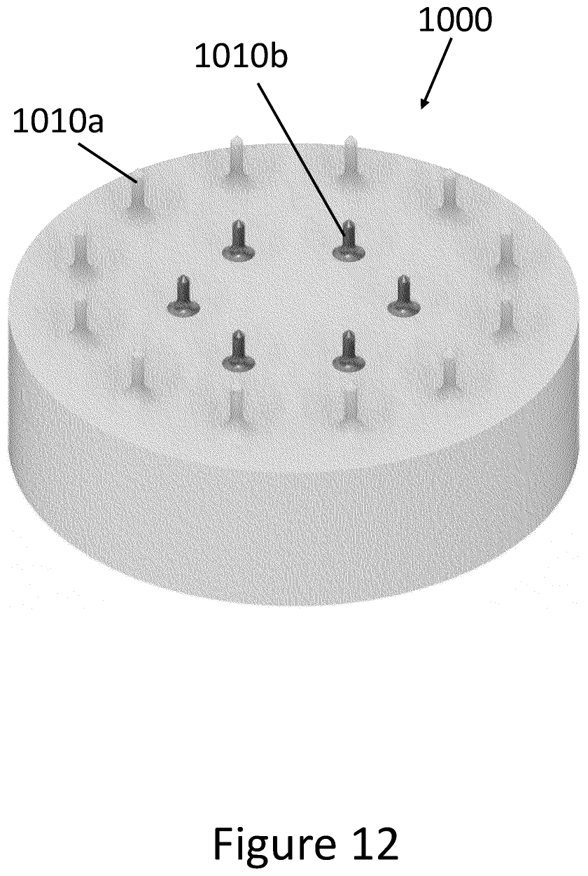

FIG. 12 shows a microneedle array device 1000. The microneedle array device 1000 comprises a first type of microneedle 1010a and a second type of microneedle 1010b. There are 12 first type microneedles 1010a which form an outer ring. There are 6 second type microneedles 1010b arranged to form an inner ring. This inner ring is enclosed by the outer ring.

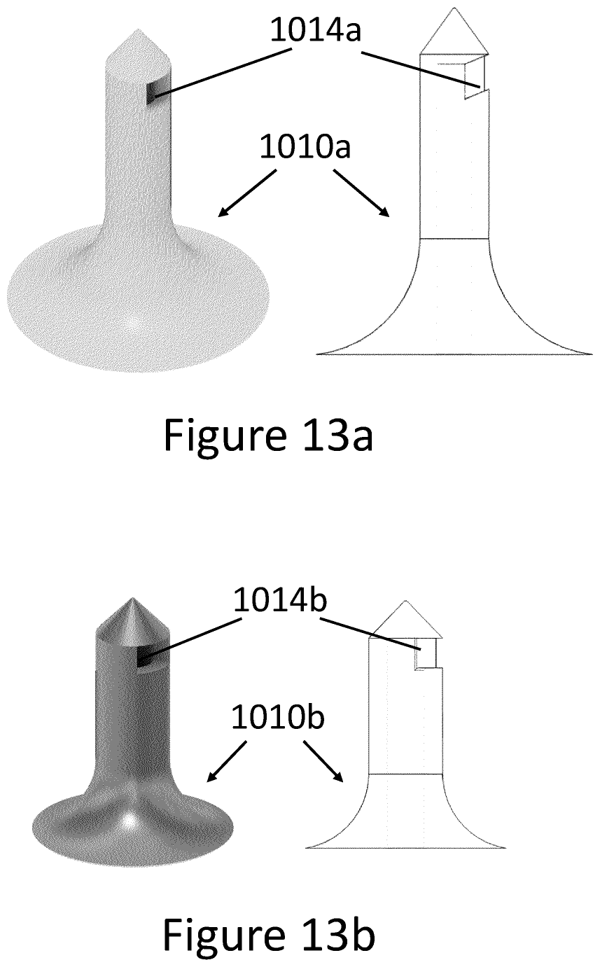

The first type of microneedle 1010a is shown in greater detail in FIG. 13a. The first type of microneedles 1010a are 1.5 mm tall and comprise a single angled outlet channel 1014a.

The second type of microneedle 1010b is shown in greater detail in FIG. 13b. The second type of microneedles 1010b are 1.0 mm tall and comprise a single outlet channel 1014b.

In the arrangement shown in FIG. 13b, the outlet channel of the second type of microneedle 1010b is angled at θ=90°. However, in other arrangements, all microneedles (both first type and second type) may have angled outlet channels that are angled at less than θ=90° and greater than θ=0°, or only the second type may have angled outlet channels angled at less than θ=90° and greater than θ=0°.

Using a microneedle array device 1000 comprising microneedle types 1010a, b with different heights permits the microneedle array device 1000 to penetrate the skin more reliably, as a patient's skin is typically rounded instead of flat.

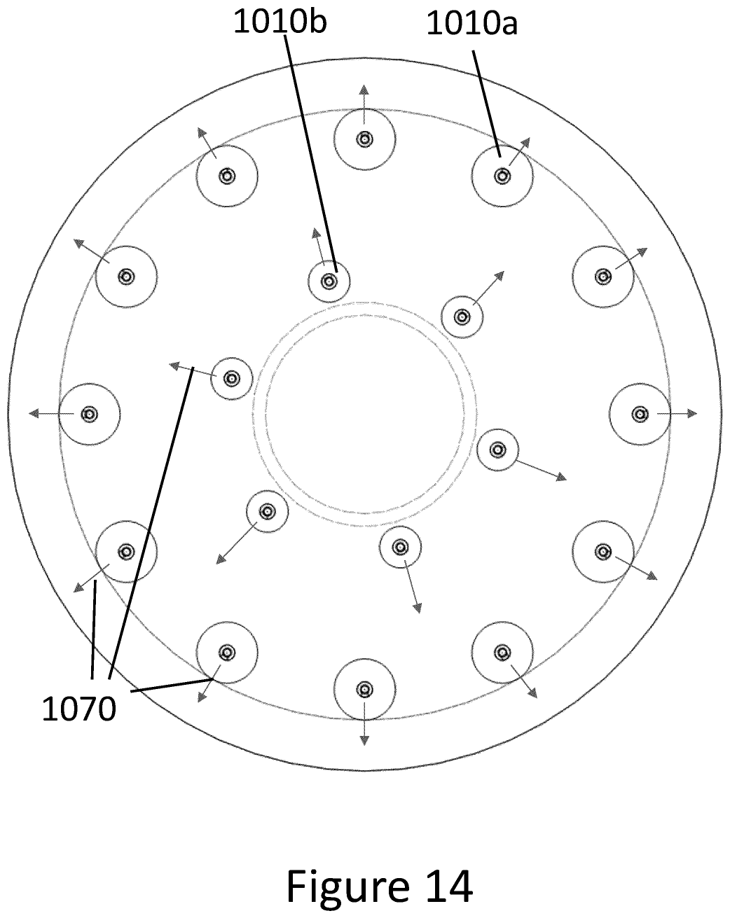

As shown in FIG. 14, each of the microneedles 1010a, b of microneedle array device 1000 are arranged such that their outlets face radially outwards, in different directions from each other. The arrows 1070 in FIG. 14 indicate the direction the outlets face for each of the microneedles 1010a, b. This design helps to minimise interference between different microneedles and diffuse drugs/fluids more evenly in the skin layer.

The lengths of the first type 1010a and the second type of microneedle 1010b are different so liquid medicaments can be delivered into different skin layers to minimise interference between microneedles to avoid fast saturation.

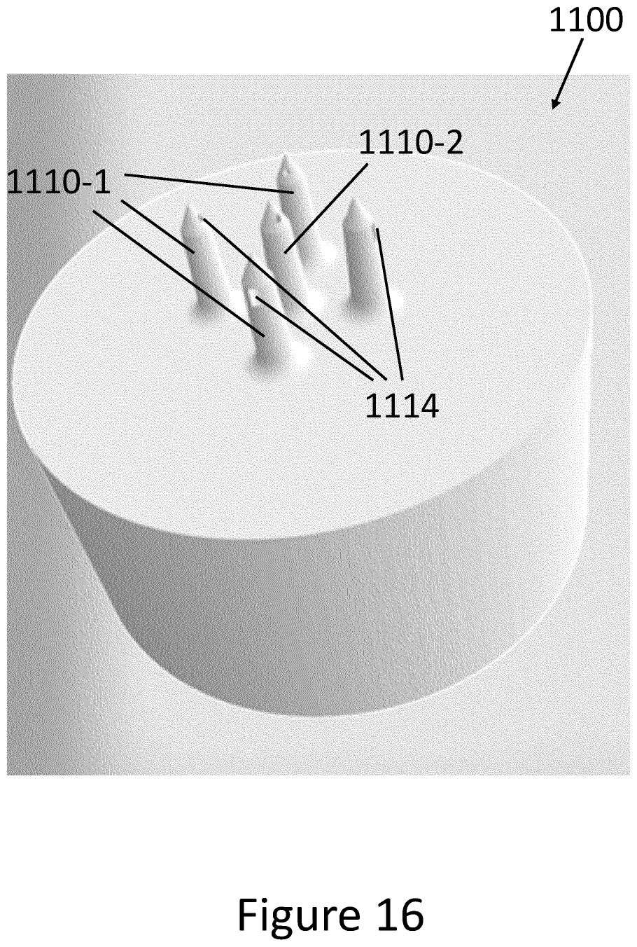

FIG. 16 shows a perspective view of a microneedle array device 1100. The device 1100 comprises five microneedle 1110. Four of the microneedles 1110-1 are arranged in a square configuration (referred to as the “outer” microneedles), with the fifth microneedle 1110-2 positioned in the centre of the other microneedles 1110-1 (referred to as the “central” microneedle).

Each of the microneedles 1110 comprises two outlet channels 1114. A first outlet channel 1114 of each microneedle 1110 is formed in the sidewall of said microneedle 1110. The first outlet channel 1114 of each of the four outer microneedles 1110-1 is arranged to face away from the other outer microneedles 1110-1. In other words, the first outlet channel 1114 of each of the outer microneedles 1110-1 is positioned on an opposing side of the microneedle 1110-1 to the central microneedle 1110-2.

A second outlet channel 1114 of each microneedle 1110 is formed in the tip of said microneedle 1110. The second outlet channel 1114 of each of the outer microneedles 1110 is arranged to face towards the central microneedle 1110.

In other embodiments, the first and/or second outlet channels 1114 may be arranged differently and face in any direction. In other embodiments, the outer microneedles 1110-1 do not form a square configuration. In other embodiments, there may be any plurality of microneedles 1110.

During experiments performed using the microneedle array device 1100 of FIG. 16, the skin of a mouse injected with medicine suffered less rupturing than when a conventional needle was used.

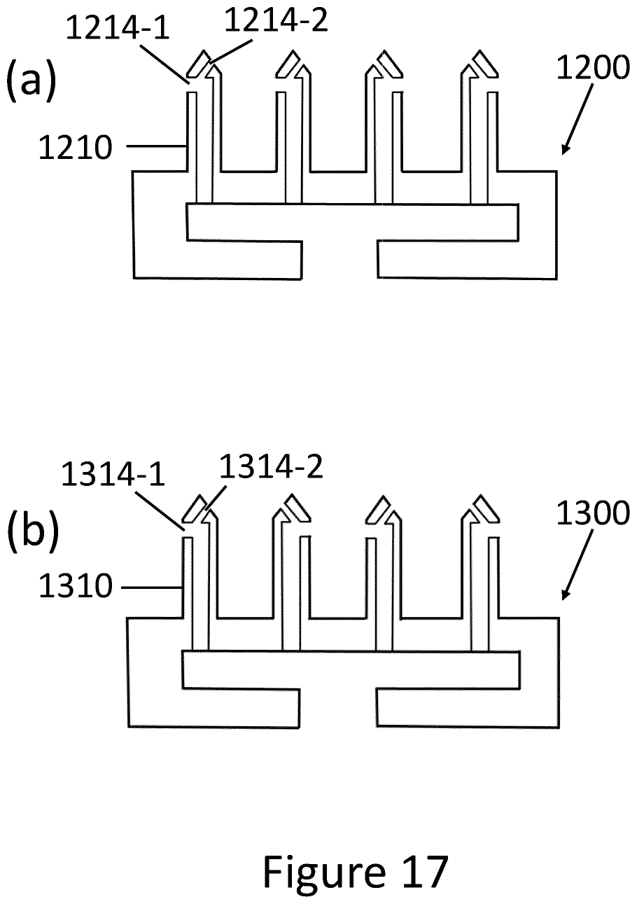

FIGS. 17(a) and (b) show microneedle array devices 1200, 1300 which comprise microneedles 1210, 1310 having outlet channels disposed in both the sidewall and the tip of the microneedle.

As shown in FIG. 17(a), microneedle 1210 comprises a first outlet channel 1214-1 disposed in the sidewall and a second outlet channel 1214-2 disposed in the tip of the microneedle 1210. In FIG. 17(a), the first outlet channel 1214-1 is angled at 90° relative to the longitudinal axis of the microneedle 1210 and the second outlet channel 1214-2 is angled at 45° relative to the longitudinal axis.

FIG. 17(a) is intended to illustrate to position and direction of the outlet channels, rather than the angles of the outlet channels. The outlet channels 1214-1 in the sidewall can be angled relative to the longitudinal axis of the microneedle at an angle which is greater than 0° and less than 90°. This is also true for FIGS. 16, 17(b), and FIGS. 18(a) and (b), and the outlet channels of those figures can also be angled relative to the longitudinal axis of the microneedle at an angle which is greater than 0° and less than 90°.

In the microneedle array device 1200 of FIG. 17(a), the first outlet channels 1214-1 all face away from the centre of the device 1200. The second outlet channels 1214-2 all face towards the centre of the device 1200.

FIG. 17(b) shows an alternative microneedle array device 1300 to FIG. 17(a), in which the outlet channels face in different directions. When compared with the embodiment of FIG. 17(a), the outlet channels of the central microneedles 1310 face the opposite direction in order to provide a different medicine delivery distribution.

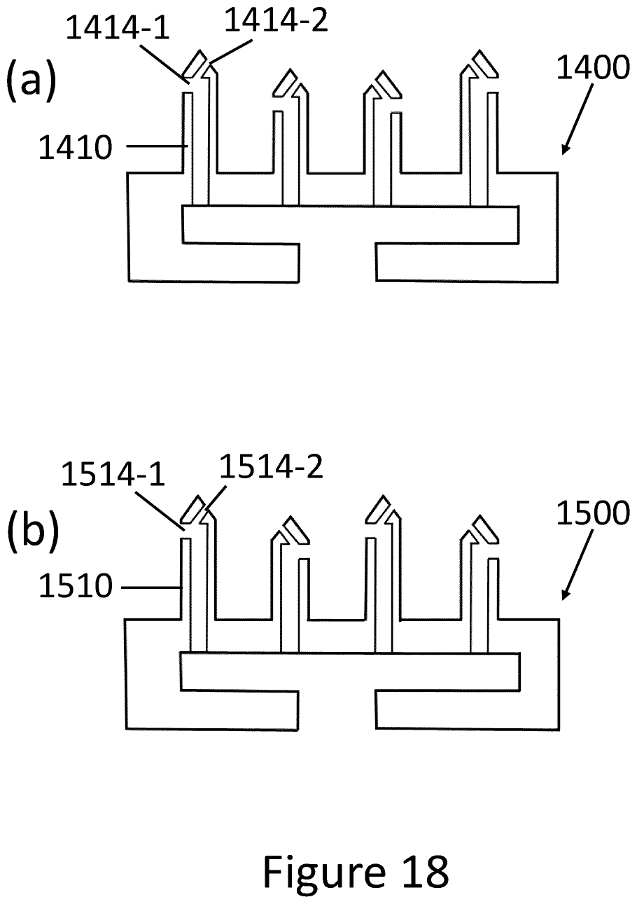

FIG. 18(a) shows a microneedle array device 1400 comprising microneedles 1410 which comprise a first outlet channel 1414-1 disposed in the sidewall and a second outlet channel 1414-2 disposed in the tip of the microneedle 1410. In the embodiment of FIG. 18(a), the first outlet channel 1414-1 is angled at 90° relative to the longitudinal axis of the microneedle 1410. The second outlet channel 1414-2 is angled at 45° relative to the longitudinal axis.

The microneedle array device 1400 of FIG. 18(a) is similar to the microneedle array device 1200 of FIG. 17(a), but the lengths of the microneedles 1410 vary. The central microneedles 1410 have a length which is shorter than the outer microneedles 1410. In other embodiments, the lengths of the microneedles can vary according to different patterns.

FIG. 18(b) shows a microneedle array device 1500 which combines the features of the microneedle array devices 1300, 1400 of FIGS. 17(b) and 18(a). The microneedle array device 1500 comprises microneedles 1510 having different lengths and with outlets facing in alternative directions—i.e., each microneedle 1510 has outlet channels facing in the opposite direction to its neighbouring microneedles 1510.

FIGS. 17(a) and (b) and FIGS. 18(a) and (b) are provided merely by way of example, and in other embodiments the length of one or more microneedles may be unique or equal to one or more of the other microneedles. The direction faced by the outlet channels of one or more microneedles may be unique, or identical to one or more of the other microneedles.

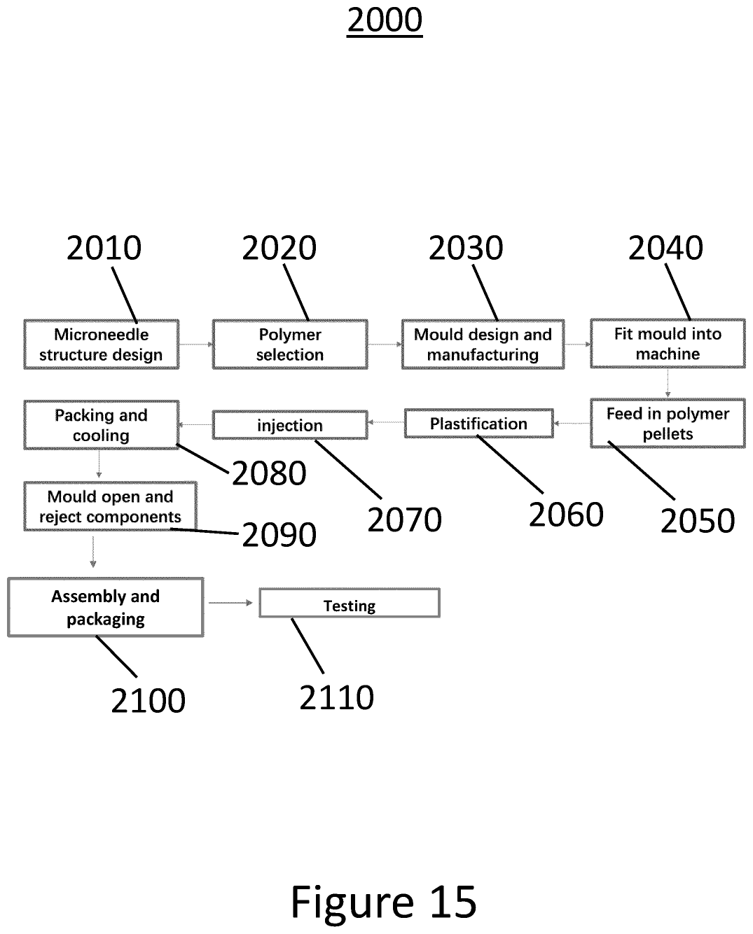

FIG. 15 shows an overview of a method 2000 of manufacturing the microneedle array device. The method 2000 comprises steps 2010-2110. The method may be used to produce the microneedles described herein and shown in FIGS. 1-6, or the microneedle array devices shown in FIGS. 7-14.

Step 2010 comprises designing the microneedle. For example, a person carrying out the method will need to decide on microneedle parameters, such as the number of outlet channels, the position(s) of the outlet channel(s), and the angle of the outlet channel(s) relative to the longitudinal axis.

Step 2020 comprises selecting a polymer to create the microneedle with. A person carrying out the method may choose from a range of high-strength bio-compatible polymeric materials. For example, the person carrying out the method may select from one or more of Polyglycolide (PGA), Polylactic acid (PLA), Polymethyl methacrylate (PMMA), Cyclic olefin copolymer (COC), Polycarbonate (PC) and Liquid crystal polymer (LCP).

Step 2030 comprises designing and fabricating the mould. The mould may be a two-piece mould. The mould may be a three-piece mould. The mould may be formed of, or comprise, one or more pieces.

In some embodiments, the mould is designed such that one or more outlet channels are formed in the microneedle during the process of injection moulding. In other embodiments, the microneedle is formed without outlet channels, and they are formed after the injection moulding process is finished. The complexity of the mould may be determined based on these factors.

Step 2040 comprises fitting the mould into an injection moulding machine. Step 2040 may comprise fitting one or more components of a mould into the injection moulding machine. In some embodiments, the injection moulding machine is a micro injection moulding machine. In some embodiments, customised modification may be made on the injection moulding machine to accommodate more complex moulds.

Step 2050 comprises feeding polymer pellets into the injection moulding machine. The pellets are formed from, or comprise, the polymeric materials selected in step 2020.

After the pellets are fed into the injection moulding machine, steps 2060 and 2070 respectively comprise plastification/melting of the pellets and injection of the melted polymeric material into the mould.

Step 2080 comprises packing and cooling the melted polymeric material within the mould. Step 2080 causes the polymeric material to solidify to form the one or more microneedle(s), and/or the base.

Step 2090 comprises opening the mould to release the one or more microneedle(s).

The method 2000 may produce a “2D string” of microneedles, referred to as a “microneedle string”, for subsequent modular assembly. For example, the mould may be designed to produce a row of microneedles. These microneedles in the same row may have the same length or different lengths. Producing a long line of microneedles reduces the complexity of the required mould, thereby reducing the time and costs associated with manufacturing compared with conventional manufacturing techniques. The microneedles can then be rearranged or assembled into a particular pattern to form 2D/3D microneedle arrays and fitted into a base to form the microneedle array device based on their desired use. Arranging the microneedles in the array is an optional step and is shown as step 2100. As a result, a manufacturer can adjust the microneedle quantity and distribution pattern in each unit by selecting suitable microneedle strings during the modular assembly, which offers more flexibility on the variation of products.

After the step 2100 of assembly and packaging, the method may comprise testing 2110 and sterilization depending on the requirements.

In some embodiments, the method 2000 produces microneedles comprising an input channel as well as one or more outlet channel(s). In order to achieve this, the mould is configured to form a microneedle comprising outlet channel(s). In other embodiments, the microneedles produced by method 2000 comprise an input channel but not outlet channels.

In some embodiments, where the injection moulding process produces microneedles without outlet channels, the method further comprises the step of removing one or more sections of the sidewall of the moulded microneedle so as to form the one or more outlet channels. Removing one or more sections of the sidewall may comprise laser cutting, direct polymer cutting, both, or other applicable manufacturing techniques.

In some embodiments, one or more of the steps 2010-2110 may be optional. For example, steps 2010-2030 may be carried out once as part of a design process, and the subsequent manufacturing steps may be repeatedly carried out to produce a large number of microneedles. This would eliminate the need for manufacturers to repeatedly select polymers and moulds.

In some embodiments, the method 2000 may be used to produce a microneedle array device, rather than one or more microneedles. In such embodiment, the microneedles will be integrally formed with a base of the microneedle array device. One or more connection means may be subsequently added to the microneedle array device. Alternatively, one or more connections means may be formed integrally with the base.

In the above examples the microneedles are shown as having a length of around 0.8 mm, 1.0 mm or 1.5 mm. However, the microneedle may have a length within the range of 0.5 to 2 mm.

From reading the present disclosure, other variations and modifications will be apparent to the skilled person. Such variations and modifications may involve equivalent and other features which are already known in the art of microneedles, microneedle array devices, or injection moulding processes, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom.

For the sake of completeness, it is also stated that the term “comprising” does not exclude other elements or steps, the term “a” or “an” does not exclude a plurality, a single processor or other unit may fulfil the functions of several means recited in the claims and any reference signs in the claims shall not be construed as limiting the scope of the claims.

1. A microneedle for transdermal drug delivery, the microneedle comprising:
an input channel extending through the microneedle along a longitudinal axis of the microneedle, the input channel defining a first end and a second end, the input channel configured to receive fluid input into the microneedle; and
one or more outlet channels, each of the one or more outlet channels defining a fluid path between the input channel and an exterior surface of the microneedle;
wherein the one or more outlet channels are angled relative to the longitudinal axis of the microneedle at an angle which is greater than 0° and less than 90°.

2. The microneedle of claim 1, wherein the input channel further defines a sidewall;
and wherein the one or more outlet channels extend between an interior surface of the sidewall, and an exterior surface of the sidewall, such that each of the one or more outlet channels define a fluid path between the input channel and an exterior surface of the sidewall.

3. The microneedle of claim 1, wherein the first end of the input channel is open, such that fluids may enter the input channel.

4. The microneedle of claim 1, wherein the second end of the input channel is closed, such that fluids cannot pass therethrough.

5. The microneedle of claim 1, wherein the angle is between 10° and 70°, and optionally or preferably, between 20° and 60°; or wherein the angle is greater than 45°.

6. (canceled)

7. The microneedle of claim 1, wherein there are a plurality of outlet channels, and the outlet channels are off-set relative to each other along the longitudinal axis of the microneedle; and/or wherein there are a plurality of outlet channels, and the outlet channels are angularly off-set relative to each other around the longitudinal axis of the microneedle, and optionally or preferably, wherein the outlet channels are angularly off-set relative to each other evenly around the longitudinal axis.

8. (canceled)

9. The microneedle of claim 1, wherein a width of the input channel varies along the longitudinal axis of the microneedle; and/or wherein the input channel has a tapered profile, a sinusoidal profile, a staggered profile, a stepped profile, or an irregular profile.

10. (canceled)

11. The microneedle of claim 1, wherein the microneedle is a cylinder, a tapered cylinder, a pyramid, a tetrahedron, or a cone.

12. The microneedle of claim 1, wherein the microneedle is formed from, or comprises, a polymeric material; and, optionally, wherein the microneedle is formed from, or comprises, high-strength bio-compatible polymeric material, such as Polyglycolide (PGA), Polylactic acid (PLA), Polymethyl methacrylate (PMMA), Cyclic olefin copolymer (COC), Polycarbonate (PC), or liquid crystal polymer (LCP).

13. (canceled)

14. The microneedle of claim 1, wherein the microneedle is formed from, or comprises, metal, ceramic, or a semiconductor material.

15. The microneedle of claim 2, wherein the one or more outlet channels comprise a first end formed in the interior surface of the sidewall, and a second end formed in the exterior surface of the sidewall, and the one or more outlet channels are tapered, such that a size of the first end of the one or more outlet channels is not equal to a size of the second end of the outlet channel.

16. A microneedle array device comprising:
a base;
one or more of the microneedles of claim 1, the one or more microneedles being disposed on a first side of the base;
a drug inlet disposed on a second side of the base; and
a hollow chamber inside the base, the hollow chamber forming a fluid connection between the one or more microneedles and the drug inlet.

17. The microneedle array device of claim 16, wherein the drug inlet comprises connection means for connecting the microneedle array device to a syringe or receptacle.

18. The microneedle array device of claim 17, wherein the connections means is, or comprises, a tapered hole; and/or wherein the connection means is, or comprises, an elastic sealing ring, or other types of sealing components.

19. (canceled)

20. The microneedle array device of claim 16, wherein the one or more of the microneedles comprise a first microneedle comprising one or more outlet channels and a second microneedle comprising one or more outlet channels, wherein the one or more outlet channels of the first microneedle are distributed in a first pattern and the one or more outlet channels of the second microneedle are distributed in a second pattern, wherein the first pattern is different to the second pattern.

21. The microneedle array device of claim 20, wherein the one or more outlet channels distributed in the first pattern have different positions along the longitudinal axis of microneedle to the one or more outlet channels distributed in the second pattern and/or wherein the one or more outlet channels distributed in the first pattern have different angular positions around the longitudinal axis of microneedle to the one or more outlet channels distributed in the second pattern.

22. (canceled)

23. The microneedle array device of claim 17, wherein there are a plurality of microneedles, and at least a first microneedle of the plurality of microneedles has a different length to at least a second microneedle of the plurality of microneedles.

24. A method of manufacturing a microneedle or a microneedle array device, according to any preceding claim the method comprising:
providing a mould;
providing a polymeric material; and
injection moulding the polymeric material using the mould to form the microneedle.

25. The method of claim 24, wherein the one or more outlet channels are formed in the step of injection moulding.

26. The method of claim 24, wherein the method further comprises the step of after the step of injection moulding removing one or more sections of a sidewall so as to form the one or more outlet channels; and preferably
wherein removing one or more sections of the sidewall comprises laser cutting, or direct polymer cutting.
